(19) United States
(12) Patent Application Publication
(10) Pub. No.: US 2018/0312911 A1
(43) Pub. Date: Nov. 1, 2018

(54) Profiling of DNA methylation using magnetoresistive biosensor array

(71) Applicants: The Board of Trustees of the Leland Stanford Junior University, Palo Alto, CA (US); Danmarks Tekniske Universitet, KGS. LYNGBY (DK)

(72) Inventors: Jung-Rok Lee, Seoul (KR); Shan X. Wang, Portola Valley, CA (US); Mikkel F. Hansen, Vaerloese (DK); Martin Dufva, Hornbaek (DK); Giovanni Rizzi, Copenhagen (DK)

(21) Appl. No.: 15/968,255

(22) Filed: May 1, 2018

Related U.S. Application Data
(63) Continuation of application No. 62/492,617, filed on May 1, 2017.

(51) Int. Cl. C12Q 1/6827; C12Q 1/6825

(52) U.S. Cl. CPC C12Q 1/6827; C12Q 2600/154; C12Q 1/6825

(57) ABSTRACT

A method of methylation detection provides a quantitative description of the methylation density in DNA strands. Bisulphite conversion [100] of the DNA strands containing methylated and unmethylated sites creates converted DNA strands with mismatch base pairs. The converted DNA strands are PCR amplified [102], and single strand target DNA strands are magnetically labeled [104] and hybridized [106] with complementary DNA strands immobilized onto a magnetoresistive (MR) sensor array. During hybridization, a binding signal may be recorded. A stringency condition such as temperature or salt concentration is increased [108] to cause the magnetically labeled single strand target DNA strands to be denatured from the complementary DNA strands immobilized onto a magnetoresistive (MR) sensor array. During the increasing of the stringency condition a denaturation signal resulting from the denatured magnetically labeled single strand target DNA strands is recorded [110] in real time and used to determine [112] stringency conditions of methylated and unmethylated DNA strands. The DNA strands may also contain wild type genes and mutated genes, so that mutation sites may be determined simultaneously with methylation sites.

### CROSS-REFERENCE TO RELATED APPLICATIONS

This application claims priority from U.S. Provisional Patent Application 62/492,617 filed May 1, 2017, which is incorporated herein by reference.

### FIELD OF THE INVENTION

The present invention relates generally to biosensing techniques and devices. More specifically, it relates to use of biosensor arrays for DNA methylation and mutation analysis.

### BACKGROUND OF THE INVENTION

Cancer is a cellular disease caused by the stepwise accumulation of genetic and epigenetic alterations. Extensive sequencing efforts have identified recurrent genetic mutations that are useful as genetic biomarkers for assessing risk of developing cancer, classifying disease subtypes, predicting response to treatment, and monitoring efficacy of treatment. DNA methylation causes epigenetic silencing of tumor suppressor genes and is studied for both its direct implication in oncogenesis and for its utility as cancer biomarker. In bladder and colon cancer, the combination of genetic and epigenetic analyses has been proven to have a higher diagnostic value than either of the two approaches applied separately. However, compared to mutation genotyping, methylation profiling is not a yes-no result, as Gene silencing mechanisms driven by methylation are generally sensitive to the overall density of methylated sites and typically multiple CpG dinucleotides (the most common methylation site) are present in gene promoters. Finally, the methylation density may vary between alleles and cells within a single tumor, resulting in a heterogeneous pattern.

A variety of techniques has been developed to detect single point mutations in DNA based on amplification, probe hybridization, enzymatic digestion, gel electrophoresis, or sequencing. DNA methylation information is lost during polymerase chain reaction (PCR) amplification, and DNA hybridization is insensitive to the methylation status of the target region. Therefore, a methylation sensitive pretreatment of the DNA has to be employed. The two main DNA methylation analysis techniques are based on methylation sensitive enzymatic digestion, affinity enrichment using antibodies specific for methylated cytosine or bisulphite conversion of unmethylated cytosine into uracil. Bisulphite conversion is most widely used since a methylation event is converted into a single base alteration (C/T) that can be detected with techniques derived from mutation detection including sequencing array hybridization, methylation sensitive PCR, and methylation sensitive melting curve analysis. Sequencing of bisulphite-converted DNA quantifies the methylation status and allows for comparison of data from different sequencing runs and batches, but it is costly and time consuming. Amplification and melting-based techniques are not specific for single methylation sites and are not easily scalable to investigate a high number of methylation sites. Array-based methods, such as the Illumina BeadChip (Illumina Inc., San Diego, Calif.), offer a highly multiplexed site-specific assay. However, after bisulphite conversion and amplification of the template, DNA products comprise mostly three bases (guanine, adenine, and thymine plus residues of methylated cytosine). This reduced sequence complexity makes design of probes for end-point detection complicated and the decreased sequence variation reduces specificity.

### BRIEF SUMMARY OF THE INVENTION

The present invention aims to perform methylation (and, optionally, mutation) profiling simultaneously in a scalable chip platform that offers highly specific and quantitative DNA methylation and mutation data on a compact, easy-to-use, and potentially low-cost platform. Our preferred approach is based on hybridization of magnetically labeled target DNA to DNA probes tethered to the surface of a GMR biosensor array. To increase the specificity of the DNA hybridization assay, we employed melting curve measurements of the surface-tethered DNA hybrids. This avoids conventional assay condition optimization since the target-probe hybrids are exposed to continuously increasing stringency during melting curve measurement. Melting curves for surface-tethered DNA probes have been also measured using fluorescence and surface plasmon resonance. Compared to these methods, the GMR biosensors offer high sensitivity, virtually no magnetic background signal from biological samples and no dependence on temperature.

In one aspect, the present invention provides a method to simultaneously profile DNA mutation and methylation events for an array of sites with single site specificity. It advantageously employs methylation detection with magnetoresistive sensor arrays, and simultaneous profiling of methylation and mutation in DNA sequences. Genomic (mutation) or bisulphite-treated (methylation) DNA is amplified using non-discriminatory primers, and the amplicons are then hybridized to an array of magnetoresistive (MR) biosensor followed by real-time melting curve measurements. This MR biosensing technique offers scalable multiplexed detection of DNA hybridization, which has been shown to be insensitive to variations in temperature, pH value and biological fluid matrix. The melting curve approach further enhances the assay specificity and tolerance to variations in probe length. Alternatively, the technique may use a method of applying methyltransferase on array to transfer methylation sites on the DNA sequences tethered to the sensor surface and directly target methylated sites for detection. This method allows for simultaneously profiling mutation and methylation sites and provides quantitative assessment of methylation density equivalent to bisulphite pyrosequencing.

Embodiments of the invention advantageously provide epigenetic and mutational analysis that may be easily implemented in a magnetic DNAchip. Magnetic detection of hybridization offers high sensitivity and virtually no magnetic background from the sample and the sample matrix. A real-time melting curve measurement of the target-probe hybrids increases the specificity of the assay by challenging the hybrids with increasingly stringent conditions. Methods to increase the stringency include, but are not limited to, raising the temperature of the MR biosensor array and decreasing the salt (Na+) concentration in the sample buffer. Importantly, the real-time melting curve measurement eliminates the need for probe optimization required for end point detection.

In one aspect, a method of methylation detection provides a quantitative description of the methylation density in DNA strands. The method includes performing bisulphite conversion of the DNA strands containing methylated and unmethylated sites to create converted DNA strands with mismatch base pairs; performing PCR amplification of the converted DNA strands to produce PCR amplified converted DNA strands; hybridizing the PCR amplified converted DNA strands to complementary DNA strands immobilized onto a magnetoresistive (MR) sensor array; magnetically labeling of the PCR amplified converted DNA strands preceding or following hybridization; increasing a stringency condition to cause the magnetically labeled single strand target DNA strands to be denatured from the complementary DNA strands immobilized onto a magnetoresistive (MR) sensor array; reading out in real time during the increasing of the stringency condition a denaturation signal resulting from the denatured magnetically labeled single strand target DNA strands; and determining stringency conditions of methylated and unmethylated DNA strands from the denaturation signal.

In one implementation, the method also includes reading out in real time a binding signal during hybridizing the magnetically labeled single strand target DNA strands with complementary DNA strands immobilized onto a magnetoresistive (MR) sensor array.

The stringency condition may be temperature, wherein increasing the stringency condition comprises increasing the temperature while salt concentration is held constant, and wherein determining the stringency conditions of the methylated and unmethylated DNA strands comprises determining melting temperatures of the methylated and unmethylated DNA strands. Alternatively, the stringency condition may be salt concentration, wherein increasing the stringency condition comprises decreasing the salt concentration while temperature is held constant, and wherein determining the stringency conditions of the methylated and unmethylated DNA strands comprises determining melting salt concentrations of the methylated and unmethylated DNA strands. Moreover, the stringency condition may be a combination of temperature and salt, wherein increasing stringency comprises simultaneously increasing temperature and decreasing salt concentration.

In any of the methods to increase stringency condition, mutation sites in the DNA may be investigated simultaneously with methylation sites. Performing PCR amplification of the converted DNA strands may include performing PCR amplification on the DNA strands after bisulphite conversion and without conversion, where the input DNA strands may contain methylated and unmethylated sites and wild type genes and mutated genes; and determining stringency conditions of methylated and unmethylated DNA strands from the denaturation signal may include determining stringency conditions of methylated and unmethylated DNA strands and wild type genes and mutated type genes from the denaturation signal, whereby mutation sites may be determined simultaneously with methylation sites.

The invention thus provides a method of methylation detection using a magnetoresistive (MR) sensor array. In preferred embodiments, DNA strands with methylated sites are bisulphite-converted and PCR amplified. They are then hybridized to a temperature-controlled magnetoresistive (e.g., GMR) biosensor array with immobilized complementary DNA strands and magnetically labeled. Target DNA strands are denatured from the immobilized DNA strands by ramping up temperature. Real-time measurements of binding signal from target DNA are used to determine melting curve. In an alternative embodiment, salt concentration rather than temperature is used for denaturing the target DNA strands. The technique can be combined with measurements of genetic mutations on the same biosensor chip.

In another aspect, a method of methylation detection provides a quantitative description of the methylation density in DNA sequences. The method includes performing bisulphite conversion of DNA strands with or without methylated sites; PCR amplification of converted DNA strands; hybridization of converted target DNA strands with a MR sensor array immobilized with (unmethylated) complementary DNA strands; adding methyltransferase to methylate the complementary DNA strands corresponding to the methylated sites of the target DNA strands; ramping up temperature until target DNA strands are denatured from the immobilized DNA strands, leaving behind the methylated single strand DNA if the target DNA is methylated, or leaving behind the unmethylated single strand DNA if the target DNA is unmethylated; adding magnetic nanoparticles conjugated with methyl-recognizing moieties, such as antimethylated lysine antibody, which will bind to methylated DNA strands immobilized on the sensor; reading out the binding signal in real time, and determining if the immobilized DNA strand (and thus the corresponding target DNA strand) is methylated or not.

Different MR technology can be used for the biosensing array. Those include, but are not limited to, giant magnetoresistive (GMR) sensors, magnetic tunnel junction (MTJ) sensors, planar Hall effect (PHE) sensors.

### BRIEF DESCRIPTION OF THE SEVERAL VIEWS OF THE DRAWINGS

FIG. 1 shows an embodiment of the invention.

FIG. 2 provides a schematic overview of an exemplary protocol for the detection of magnetically labeled DNA using a GMR biosensor device.

FIG. 3A is a graph of the real-time monitoring of ΔMR signal from GMR biosensors.

FIGS. 3B-C show melting curves from wild type WT and mutant type MT probes targeting BRAF c. 1391 G>A mutation.

FIGS. 4A-B are schematic illustrations of the bisulphite conversion process.

FIGS. 4C-D show melting curves from methylated (M) and unmethylated (U) probes targeting KIT methylation (site p1).

FIG. 5A shows mutation profiling of melanoma cell lines.

FIGS. 5B-C are a heat map and a mutation map, respectively, corresponding to FIG. 5A.

FIGS. 6A-C show results of mutation and methylation profiling of melanoma cell lines.

FIG. 7A is an exemplary schematic diagram of a differential magnetoresistive sensor bridge.

FIG. 7B is a schematic representation of temperature and salt concentration melting.

FIG. 7C is an exemplary schematic measurement setup.

FIGS. 8A-B show the WT target melting curves measured for c(Na⁺)=10 mM and 2 mM, respectively of MNP labeled WT DNA target hybridized to WT and MT DNA probes for the CD8/9 mutation.

FIGS. 9A-B show salt concentration melting curves of WT DNA target hybridized to WT and MT DNA probes for the CD8/9 mutation.

FIG. 10 shows the values of melting temperature Tₘ and salt concentration cₘ for the WT target and WT probes (filled symbols) and MT probes (open symbols) for the CD 8/9 locus of HBB.

FIG. 11A shows the temperature melting curve measured at c(Na⁺)=10 mM for the CD 8/9 locus.

FIG. 11B shows the salt concentration melting curve measured at T=37° C. for the CD 8/9 locus.

FIGS. 11C-D show the corresponding temperature and salt concentration melting profiles measured for the CD 17 locus.

### DETAILED DESCRIPTION OF THE INVENTION

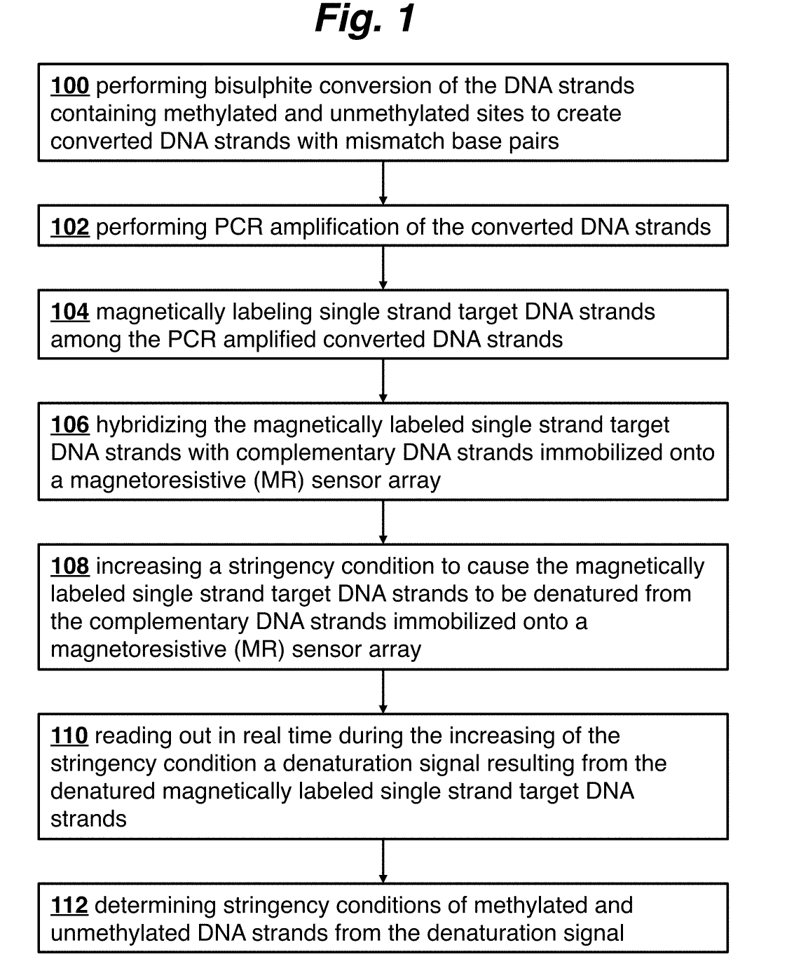

FIG. 1 is a flow chart providing an overview of a method of methylation detection providing a quantitative description of the methylation density in DNA strands, according to an embodiment of the invention. In step 100, bisulphite conversion of the DNA strands containing methylated and unmethylated sites is performed to create converted DNA strands with mismatch base pairs. In step 102, PCR amplification of the converted DNA strands is performed. In step 104, single strand target DNA strands among the PCR amplified converted DNA strands are magnetically labeled. In step 106, the magnetically labeled single strand target DNA strands are hybridized with complementary DNA strands immobilized onto a magnetoresistive (MR) sensor array. In some implementations, during hybridizing a binding signal is read out in real time.

In step 108, a stringency condition is increased to cause the magnetically labeled single strand target DNA strands to be denatured from the complementary DNA strands immobilized onto a magnetoresistive (MR) sensor array. In step 110, during the increasing of the stringency condition a denaturation signal resulting from the denatured magnetically labeled single strand target DNA strands is read out in real time. In step 112, stringency conditions of methylated and unmethylated DNA strands are determined from the denaturation signal.

The stringency condition may be temperature, in which case increasing the stringency condition comprises increasing the temperature while salt concentration is held constant. Determining the stringency conditions of the methylated and unmethylated DNA strands in this case comprises determining melting temperatures of the methylated and unmethylated DNA strands. Alternatively, the stringency condition may be salt concentration, in which case increasing the stringency condition comprises decreasing the salt concentration while temperature is held constant. Determining the stringency conditions of the methylated and unmethylated DNA strands in this case comprises determining melting salt concentrations of the methylated and unmethylated DNA strands.

In some implementations, the method may be used to determine mutation sites simultaneously with methylation sites. In this case, performing bisulphite conversion of the DNA strands containing methylated and unmethylated sites includes performing bisulphite conversion of the DNA strands containing methylated and unmethylated sites and wild type genes and mutated genes. In addition, determining stringency conditions of methylated and unmethylated DNA strands from the denaturation signal in this case includes determining stringency conditions of methylated and unmethylated DNA strands and wild type genes and mutated type genes from the denaturation signal.

The method described above will now be illustrated by way of several examples. The following abbreviations will be used: GMR, Giant Magnetoresistive; KIT, Tyrosine Kinase; MNP, Magnetic NanoParticle; MR, MagnetoResistance; MT, Mutant Type; PCR, Polymerase Chain Reaction; RARB, Retinoic Acid Receptor β; SNP, Single Nucleotide Polymorphism; WT, Wild Type.

### DNA Mutation Analysis

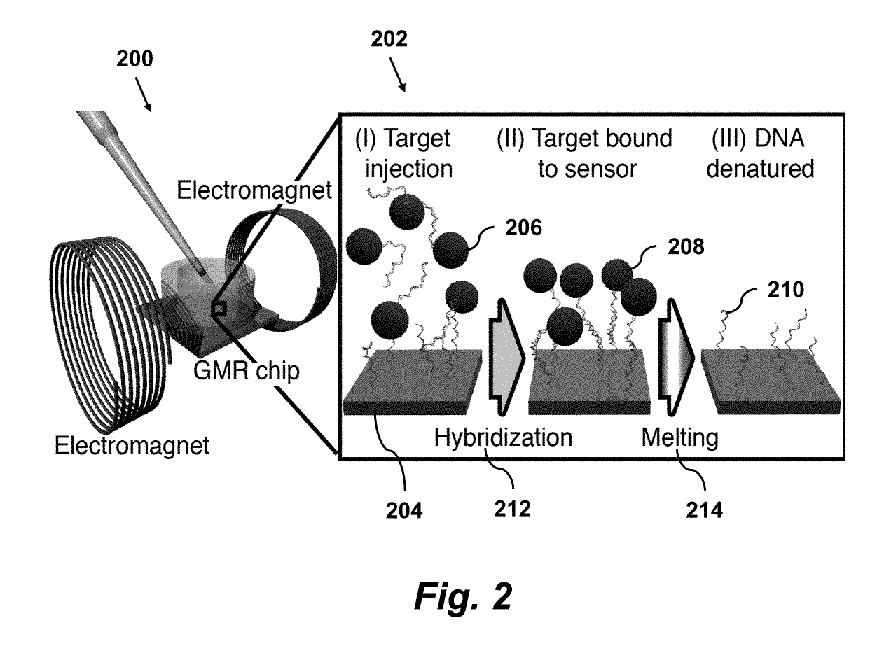

FIG. 2 provides a schematic overview of a protocol 202 for the detection of magnetically labeled DNA using a GMR biosensor device 200, according to an embodiment of the invention. After denaturation of the reverse strand and labeling, PCR products are injected into the reaction well over the chip 204. In a hybridization step 212, DNA labeled with MNPs 206 hybridizes to complementary surface-tethered probes for 1 hour at 37° C., resulting in hybridized DNA labeled with MNPs 208. Unbound sample is removed by washing. In melting step 214, the temperature is swept from 20° C. to 65° C., causing denaturization of the DNA from the probes 210 to measure the melting temperature, Tₘ.

To detect DNA mutations, we PCR amplified the genomic regions of interest using non-discriminatory primers. The PCR products were then magnetically labeled using biotinylated primers and streptavidin-coated magnetic nanoparticles (MNPs). After magnetic column separation and denaturation of the double-stranded PCR products, ssDNA conjugated to MNPs (MNP-ssDNA) was introduced to the GMR biosensor array where multiple DNA probes were separately tethered to the surface of each sensor. Upon hybridization of the injected MNP-ssDNA to surface-tethered complementary probes, GMR biosensors produced changes in sensor magnetoresistive ratio (ΔMR) proportional to the bound MNPs. To genotype a mutation, we employed a set of two probes complementary to the wild type (WT) and mutant type (MT) sequences of the sample (supplementary information Table 1). During hybridization at low stringency, amplicons hybridized to both WT and MT probes with similar affinity. To obtain single base specificity, stringent washing is typically used after hybridization in DNA microarray. To achieve a more flexible system for detection of single-base mutations, we challenged the hybrids by increasing the temperature and continuously measuring DNA melting simultaneously for all probes on the GMR biosensor array.

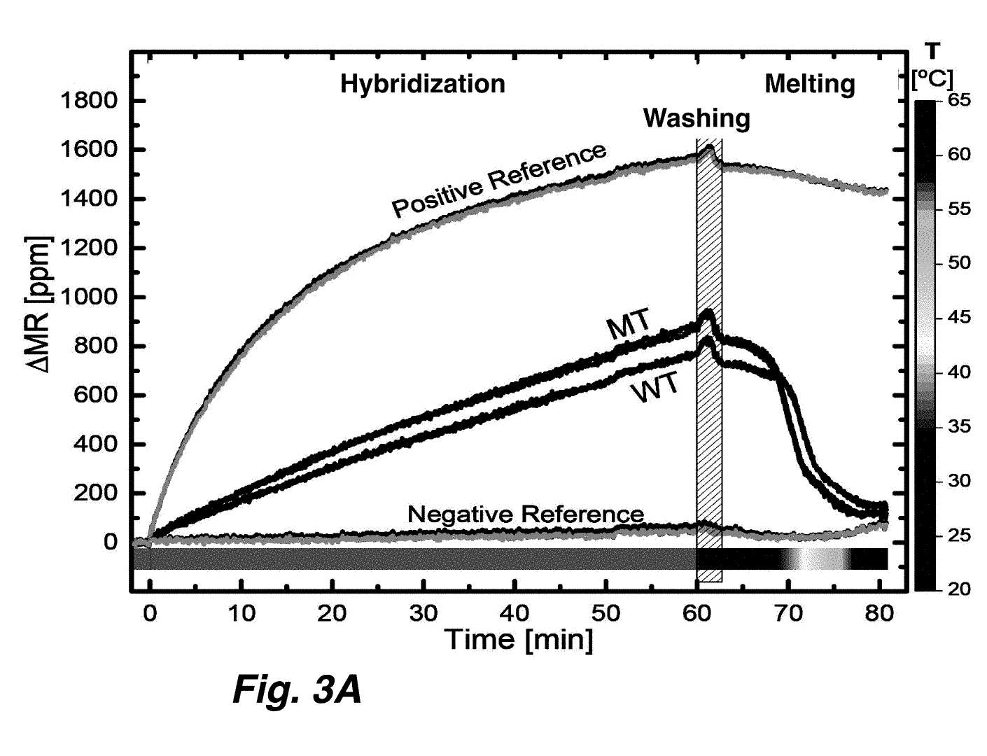

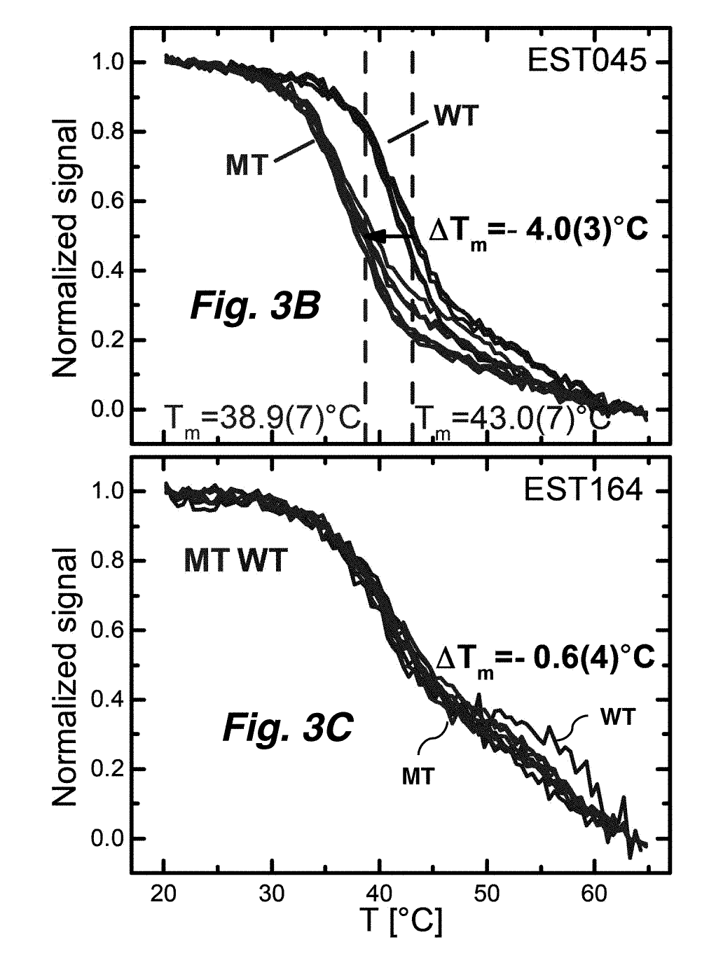

FIG. 3A is a graph of the real-time monitoring of ΔMR signal from GMR biosensors functionalized with positive and negative references, wild type (WT) and mutant type (MT) probes for the BRAF c.1391G>A mutation. The signal was measured during hybridization (1 hour at 37° C.) of a known WT sample to probes with a perfect match (WT) or a single-base mismatch (MT) for the BRAF c.1391G>A mutation. Each line corresponds to up to three sensors functionalized with the same probe. The measurement was performed with PCR products from EST045 cell line that is WT for the investigated mutation. The sample was injected at t=2 min.

In addition, a biotinylated DNA probe was used as positive reference and a DNA probe with an unspecific sequence was used as negative reference. After 1 hour of hybridization, the MT probe gave a slightly higher ΔMR signal than the WT probe, indicating that low-stringency hybridization was insufficient to genotype the WT sample.

After 60 min hybridization at 37° C., the unbound sample was removed by a low-temperature wash at low stringency. Then, the temperature was ramped from 20° C. to 65° C. at constant rate while measuring the melting curves until all DNA hybrids melted. The signal (ΔMR) from GMR biosensors was corrected for its temperature dependence during ramping using the sensor resistance (R), which is linearly related to the sensor temperature. FIG. 3B and FIG. 3C show melting curves from WT and mutant type MT probes targeting BRAF c.1391G>A mutation obtained for the indicated cell lines, where the EST045 and EST164 cell lines were wild type and homozygous mutant, respectively. Signals were normalized by the initial signal at T=20° C. The melting temperature Tₘ is defined as the temperatures at which the normalized curves cross 0.5. ΔTₘ is the difference in melting temperature between the MT and WT probes. The numbers in parentheses are standard deviations on the last significant digit (n=4-6).

FIG. 3B shows the melting curve of WT BRAF amplicons hybridized to WT and MT probes for the c.1391G>A mutation. Here, the ΔMR signal was normalized by the initial signal at T=20° C. We defined the melting temperature Tₘ as the temperature at which the signal (ΔMR) dropped to the half of its initial signal (at 20° C.). Each melting experiment was repeated with two identical GMR biosensor chips. Three sensors were functionalized with each probe, thus generating up to six identical melting curves for each probe. The obtained melting curves were found to be highly reproducible—both from sensor to sensor and from chip to chip. The hybrids of the target DNA with WT and MT probes in FIG. 3B showed melting temperatures of Tₘ(WT)=43.0(7) ° C. and Tₘ(MT)=38.9(7) ° C., respectively, where the numbers in parentheses are standard deviations of Tₘ on the last digit (n≥4). We defined the melting temperature difference, ΔTₘ, as the difference between the melting temperature from the MT probe and that from the WT probe, ΔTₘ=Tₘ(MT)−Tₘ(WT). Thus, ΔTₘ<0 indicates a higher complementarity of the target to the WT probe than the MT probe, and hence that the target is WT. The obtained value ΔTₘ=−4.0(3) ° C. is in agreement with the expectation for a single base mismatch between the WT target and MT probe using a nearest neighbor calculation.²⁸ We also note that the lower standard deviation of ΔTₘ compared to Tₘ indicates that differences in melting temperatures were more reproducible than their absolute values.

FIG. 3C shows melting curves measured for a cell line heterozygous for the BRAF c.1391G>A mutation.²² The melting curves from WT and MT probes were found to overlap each other, resulting in ΔTₘ=−0.6(4) ° C. because the heterozygous sample contains both MT and WT targets, which hybridize to both WT and MT probes. The resulting melting curves from WT and MT probes were both given by the contribution of low-Tₘ and high-Tₘ DNA hybrids. Therefore, the melting curves overlapped and presented a lower slope.

### DNA Methylation Analysis

We applied a similar detection scheme to analyze the methylation state of specific regions of the target. We employed bisulphite treatment of the genomic DNA to convert a methylation event into a single base substitution (C>T). After bisulphite conversion, we amplified the gene promoter region of interest by non-discriminatory PCR.

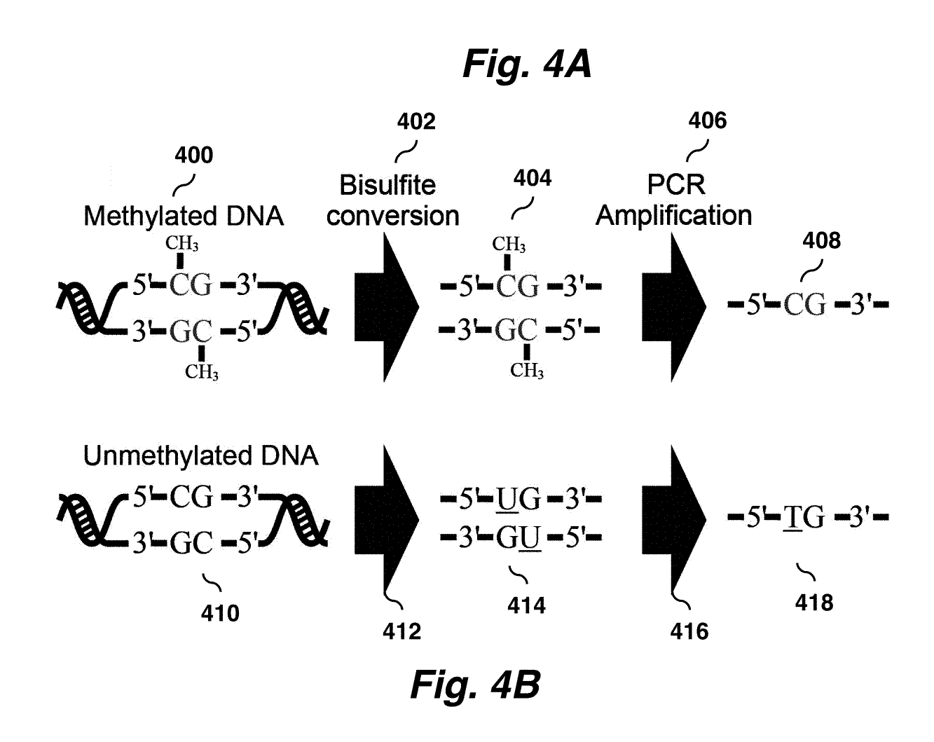

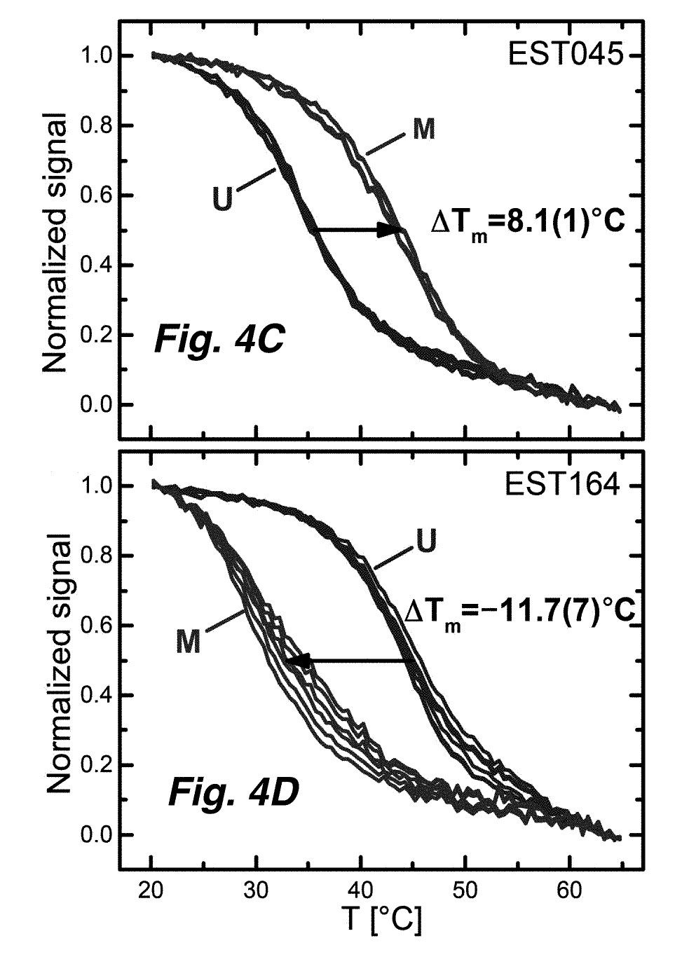

FIG. 4A, FIG. 4B are schematic illustrations of the bisulphite conversion process. Upon bisulphite treatment 402, 412, unmethylated cytosines in DNA 410 are converted to uracil in DNA 414 (FIG. 4B) whereas 5-methylcytosines in DNA 400 are retained in DNA 404 (FIG. 4A). In the subsequent PCR 406, 416, which produces products 408, 418, uracil in 414 is substituted by thymine. Thus, the methylated cytosines are mapped to single base alterations (C>T) of the amplicons.

FIG. 4C and FIG. 4D show melting curves from methylated (M) and unmethylated (U) probes targeting KIT methylation (site p1). The melting curves were measured for FIG. 4C the hypermethylated cell line EST045 and FIG. 4D the unmethylated cell line EST164. The melting curves are used to estimate methylation status of the KIT promoter (site p1) of hypermethylated (EST045) and wild-type (EST164) cell line. The amplicons were hybridized to probes complementary to unmethylated (U) or methylated (M) target DNA. Melting curves were measured as described previously. Here, ΔTₘ was defined as the melting temperature of the M probe minus that of the U probe, ΔTₘ=Tₘ(M)−Tₘ(U). Thus, a negative ΔTₘ indicates a higher complementarity of the target to the U probe and a lower degree of methylation. The ˜20 bp region of the KIT promoter investigated includes three CpG sites that can be methylated (sequences in supplementary information Table 1), and thus we expect higher ΔTₘ than for single base substitution. For the hypermethylated cell line in FIG. 4C, we found ΔTₘ=8.1(1) ° C., confirming the hypermethylation status of the KIT promoter, whereas we found ΔTₘ=−11.7(7) ° C. for the WT cell line in FIG. 4D, indicating the unmethylated status.

### Multiplex DNA Profiling of Melanoma Cell Lines

The GMR biosensor array comprises of 64 individual sensors that can be individually functionalized with amino-modified DNA probes. Using the mutation and methylation detection techniques described above, we simultaneously probed three mutation sites in BRAF, two mutation sites in NRAS, two methylation sites in the KIT promoter, and two methylation sites in the RARB promoter in triplicate. We performed mutation and methylation profiling of seven melanoma cell lines. For each cell line, the targeted regions of BRAF and of NRAS were amplified by non-discriminatory PCR. Also, the promoter regions of KIT and RARB were amplified by non-discriminatory PCR after bisulphite conversion. After magnetic labeling, a mixture of all amplicons from a cell line was injected over the sensor surface. For each cell line, melting curve profiling was repeated with two nominally identical GMR biosensor arrays. The melting curves were analyzed in terms of melting temperatures, and we determined ΔTₘ for all investigated mutations and methylation.

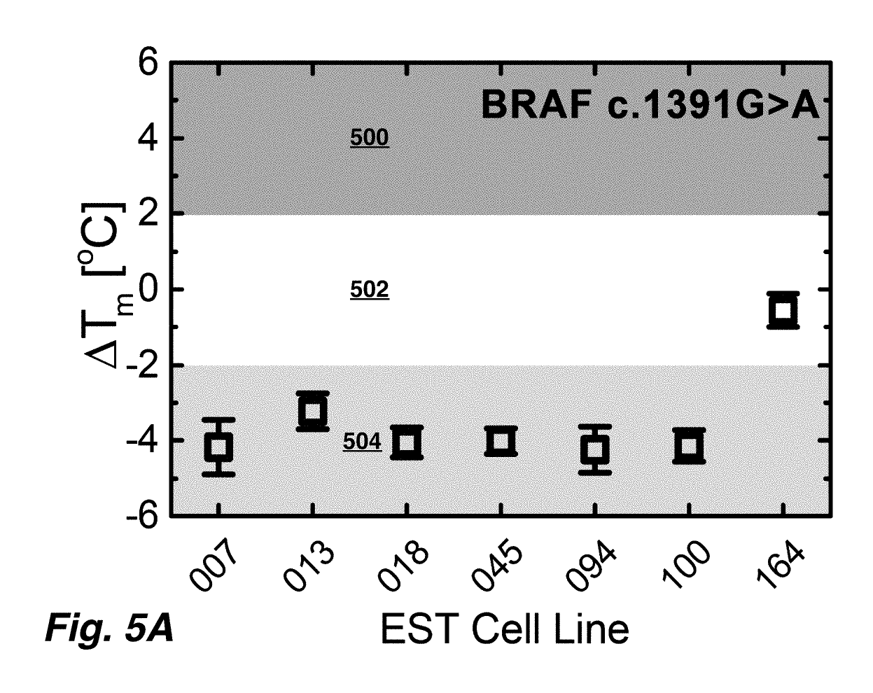

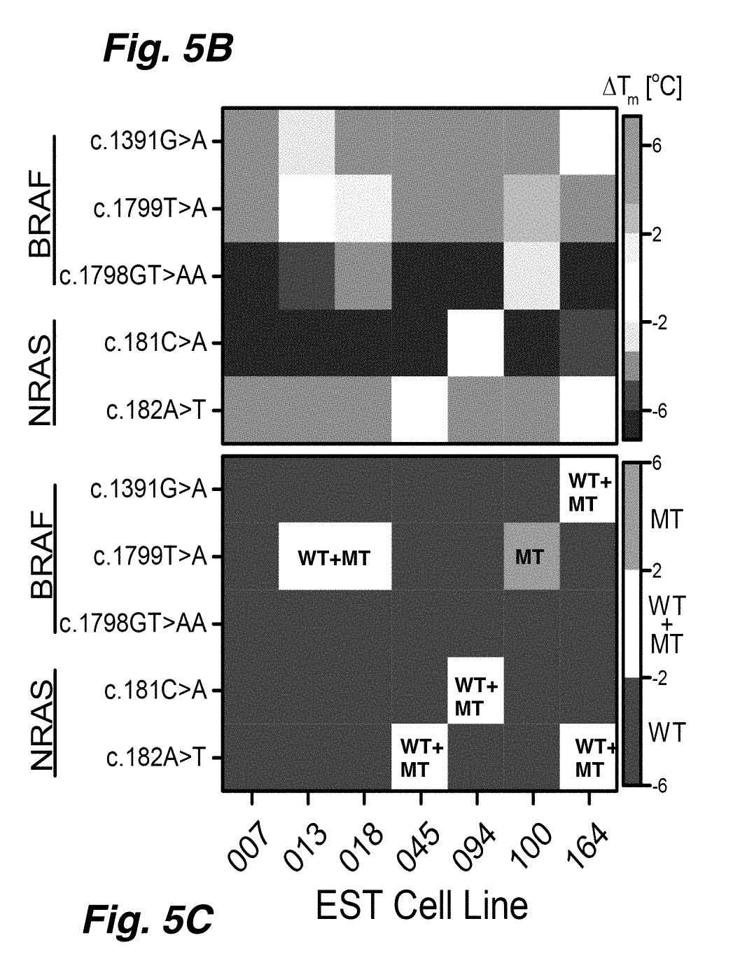

FIG. 5A Mutation profiling of melanoma cell lines. ΔTₘ measured for BRAF c.1391G>A mutation for the seven investigated EST cell lines. Error bars are one standard deviation (n=4-6). The horizontal lines are threshold values used for genotyping: ΔTₘ<−2° C. WT, 2° C.<ΔTₘ<2° C. heterozygous MT, ΔTₘ>2° C. homozygous MT. FIG. 5B Heat map of ΔTₘ measured for the mutation and for the investigated EST cell lines. FIG. 5C Heat map of measured ΔTₘ with applied threshold to genotype mutations.

FIG. 5A shows the ΔTₘ values measured for the BRAF c.1391G>A mutation for all cell lines. Six cell lines showed ΔTₘ values around ΔTₘ=−4° C., indicating a homozygous WT sequence. EST164 is known to be the only cell line with a heterozygous mutation in this site, showing ΔTₘ=−0.5(4) ° C., which is significantly different from the other cell lines.

The ΔTₘ values measured for all investigated mutations for each cell line are displayed in the heat map of FIG. 5B. Classifying WT (ΔTₘ<−2° C.), heterozygous MT (−2° C.<ΔTₘ<2° C.), and homozygous MT (ΔTₘ>2° C.) resulted in the mutation map presented in FIG. 5C. All mutations identified in the cell lines were consistent with previous genotyping data. For the NRAS c.182 A>T mutation in the cell line EST045, we measured ΔTₘ=−0.2(4) ° C., genotyping the cell line as heterozygous for this mutation; however, the cell line is known to be heterozygous for an A>G substitution in that location. As an MT probe targeting an A>T mutation was employed, both the WT and MT probes were similarly mismatched to the target, resulting in ΔTₘ close to zero. The absolute values of Tₘ were comparable to the other investigated mismatched probes confirming the mismatch of the target to both the WT and MT probes. Therefore, an unknown mutation can be detected by a lower Tₘ from the WT probe, but probes targeting all possible mutations should be included in the assay to perform accurate genotyping.

### DNA Methylation Density

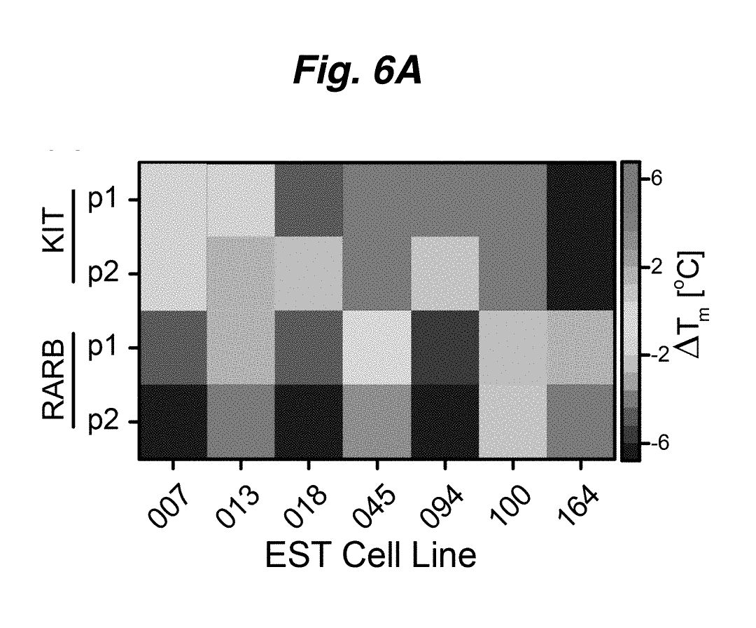

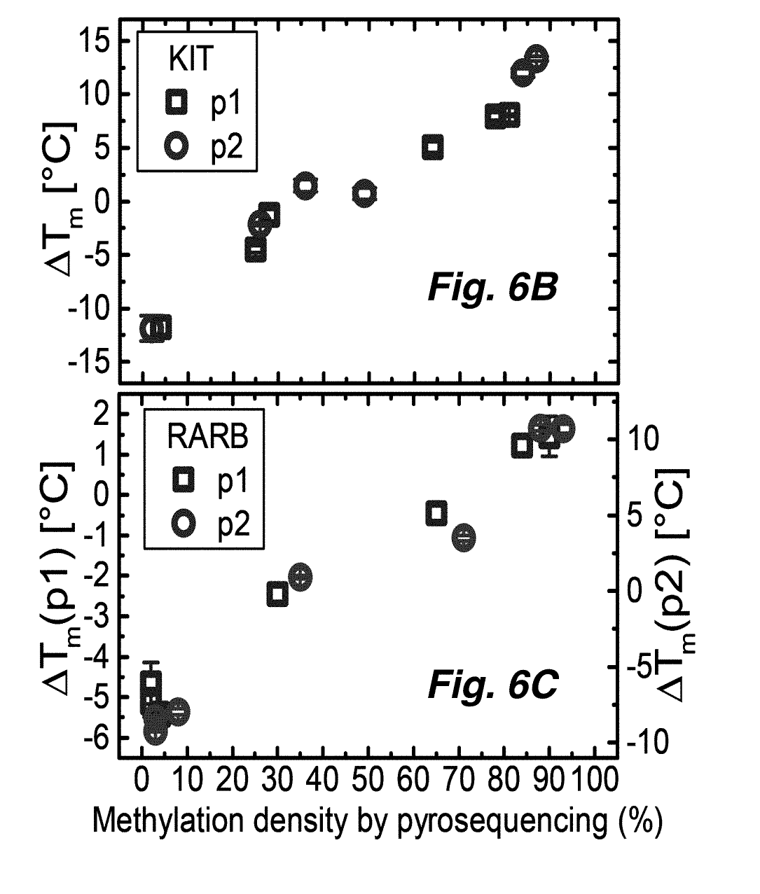

Methylation profiling differs substantially from genotyping since the methylation status of each CpG site in the promoter region varies between alleles and within a cell population. Therefore, it requires a different data analysis in terms of methylated fraction of the sample DNA. We measured melting curves using surface-tethered probes targeting two locations of the KIT promoter and two locations of the RARB promoter. The targeted sequences contain one to four CpG sites. Combining multiple investigated sites with the intrinsic variation of the methylation pattern, we obtained a continuous variation of ΔTₘ for the analyzed cell lines. FIG. 6A shows the measured ΔTₘ values for all cell lines. Here, higher ΔTₘ indicates higher affinity of the sample to the M probe, i.e., a hypermethylation event. The complex ΔTₘ pattern is a direct consequence of the intrinsic methylation variation.

FIG. 6A-C show results of mutation and methylation profiling of melanoma cell lines. FIG. 6A is a heat map of ΔTₘ measured for KIT and RARB methylation probes for the seven investigated EST cell lines. Calculation of ΔTₘ for EST007 KIT was not possible due to low binding signal. FIG. 6B is a graph of ΔTₘ values measured for KIT p1 (squares) and p2 (circles) methylation probe locations vs. methylation density measured by pyrosequencing. FIG. 6C is a graph of ΔTₘ measured for RARB p1 (squares) and p2 (circles) methylation probe locations vs. methylation level measured by pyrosequencing. Error bars are one standard deviation (n=4-6).

The methylation density was assessed independently by pyrosequencing of the bisulphite-converted DNA. To each target sequence corresponding to the probes, we calculated methylation density depending on both the fraction of methylated sample and the number (1 to 4) of methylated CpG sites in the region targeted by the probe. FIG. 6B, Fig. C show ΔTₘ measured using the GMR biosensor versus the methylation density obtained by pyrosequencing for the KIT p1 and p2 probe locations and the RARB p1 and p2 probe locations, respectively. In these plots, each point corresponds to one of the measured cell lines. There is an evident linear correlation between ΔTₘ and the methylation density (R²>0.94 for all probe locations, results of linear regression are given in supplementary Table 4). For the KIT p1 and p2 probes, the slopes are comparable (˜0.22° C./%, FIG. 6B), whereas for the RARB probes, the slopes for the p1 and p2 probes differ significantly (p1: 0.076(5) ° C./%, p2: 0.22(2) ° C./%). The slope for the p2 probe was three times that for the p1 probe because the p2 probe covers three CpG sites whereas the p1 probe only covers one CpG site. Nevertheless, three methylation sites allowed for a more complex pattern of methylation sites and thus the RARB p2 probe showed a broader spread of data around the best linear fit. The probes can be tailored to sacrifice linearity to favor higher values of ΔTₘ.

These results demonstrate the application of real-time temperature melting on a GMR biosensor as a novel and quantitative method for profiling methylation density. High-throughput profiling of genome wide methylation can be performed with single-base resolution using array-based methods like Illumina BeadChips but the specificity of such arrays is limited by lower sequence variability of bisulphite converted DNA. A quantification of the overall methylation density of a gene promoter can be obtained with methylation-specific melting curve analysis. Here, we combined the throughput and scalability of arrays with the specificity and flexibility of melting curve analysis. The obtained quantitative profiling was equivalent to the results of pyrosequencing.

The above examples have illustrated an approach for simultaneous DNA mutation and methylation profiling. Our method combines the DNA microarray techniques for both mutation and methylation analysis in a single platform. Melting curves measurements are used to increase the specificity of mutation detection. For methylation detection, the melting curve quantifies the methylation state at a level equivalent to pyrosequencing. The same technique could potentially be employed on a variety of other platforms capable of real-time monitoring of the DNA hybridization vs. temperature.

The GMR biosensor platform has a low cross-sensitivity to temperature and provides a sensitive readout. Although it does not offer the extreme throughput as advanced bead microarray systems (e.g.: Illumina), in its present format, the GMR biosensor platform can be used for the simultaneous triplicate investigation of about 20 mutation and methylation sites. This number is sufficient for many clinical applications where focus is limited to a small number of mutations and methylation sites of relevance for a specific cancer. Nevertheless, the GMR biosensor array has a modular design that can be scaled to include up to thousands of biosensors.²⁷

### Methods

### Cells and Reagents.

Melanoma cell lines for this study were obtained from The European Searchable Tumour Line Database (ESTDAB: http://www.ebi.ac.uk/ipd/estdab) and were maintained in RPMI-1640 medium containing 10% FBS and antibiotics at 37° C. and 5% CO₂. The PCR primers for this study have been modified from Dahl et al. and were obtained from DNA Technology A/S, Denmark. The sequences can be found in the supplementary material Table 2. The amine modified DNA probes (sequences in supplementary material Table 1) were matched for melting temperature calculated with nearest-neighbor model. The probes were obtained from DNA Technology A/S. The other reagents: poly(ethylene-alt-maleic anhydride) (Sigma Aldrich), poly(allylamine hydrochloride) (Polyscience), distilled water (Invitrogen), 1-Ethyl-3-(3-dimethylaminopropyl)-carbodiimide EDC (Sigma Aldrich), N-hydroxysuccinimide NHS (Sigma Aldrich) 1% bovine serum albumin BSA (Sigma Aldrich), phosphate buffered saline PBS (Gibco), Tween 20 (Sigma Aldrich), Urea (Fisher Scientific), 20× saline sodium citrate SSC (Invitrogen), mineral oil (Sigma Aldrich), MNPs Streptavidin MicroBeads (Miltenyi), magnetic separation columns μ Columns (Miltenyi).

### DNA Extraction and Bisulphite Treatment.

Genomic DNA was isolated using the Qiagen AllPrep DNA/RNA/Protein Mini kit (Qiagen GmbH, Hilden, Germany) and quantified using a NanoDrop ND-1000 spectrophotometer (NanoDrop Technologies, Wilmington, Del.). Bisulphite conversion of DNA (500 ng) was carried out using the EZ DNA Methylation-Gold™ Kit (Zymo Research, Irvine, Calif.) according to the manufacturer's protocol.

### PCR Amplification.

Prior to the GMR biosensor assay, PCR was performed using a Veriti™ 96-Well Thermal Cycler (Applied Biosystems) and TEMPase Hot Start Polymerase (VWR). All amplifications were initiated with enzyme activation and DNA denaturation at 95° C. for 15 minutes, 40 cycles of 95° C. for 30 seconds, 56° C. for 30 seconds and 72° C. for 30 seconds, followed by a final incubation at 72° C. for 10 minutes. All primer sequences used are listed in the supplementary material Table 2.

### Magnetic Labeling.

Products from PCR amplification of each cell line were processed as described by Rizzi et al. to obtain ss-DNA target conjugated with MNPs. Briefly, for each amplified region, 10 μL of PCR products were mixed with 10 μL of stock solution of streptavidin-coated MNPs (MACS Streptavidin Microbeads, cat: 130-048-102, Miltenyi Biotec Norden AB, Lund, Sweden) and incubated for 30 min at 37° C. A magnetic separation column (μ column, Miltenyi Biotec Norden AB, Lund, Sweden) was prepared by washing with 1 mL of 1% Tween 20 and 1 mL of 0.1% BSA containing 0.05% Tween 20, sequentially. After the conjugation with magnetic particles, the five PCR products were mixed and added to the column under an applied magnetic field for separation. While the target DNA-bead complexes were trapped in the column, the reverse strands were denatured and removed by adding 2 mL of 6 M Urea solution at 75° C. Then, the applied magnetic field was removed, and the conjugated complexes were eluted with 100 μL of 2×SSC buffer.

### Sensor Preparation.

The GMR biosensor chip with an array of 8×8 sensors was fabricated as previously described. The chip surface was chemically activated following Kim et al. Briefly, the chip was sequentially washed with acetone, methanol, and isopropanol. After cleaned with oxygen plasma, the chip was treated with poly(ethylene-alt-maleic anhydride) for 5 min. Then, the chip was washed with distilled water, and baked at 110° C. for 1 hour using a hot plate. After treatment with poly(allylamine hydrochloride) for 5 min, the chip was washed with the distilled water, and activated with a mixture of NHS and EDC for 1 hour. After the chip was washed again with distilled water, a robotic arrayer (sciFlexarrayer, Scienion) was used to print the amino-modified DNA probes on different sensors (Supplementary Table 2). Each DNA probe was dissolved in 3×SSC buffer at 20 μM, and was used for printing in triplicate. Four sensors on the same chip were functionalized with biotinylated DNAs as positive references, and another set of four sensors was functionalized with DNA non-complementary to any of the PCR amplified regions as negative references. The chip was stored at room temperature until use.

### Data Acquisition for Temperature Calibration.

Prior to the assay, the thermal resistivity of each GMR sensor was characterized for temperature calibration. The temperature coefficient for each chip was obtained by linear fitting to resistance measurements at 20, 30, 40, 50 and 60° C. The temperature coefficient was then used to trace the instantaneous temperature of each sensor.

### GMR Biosensor Assay.

The measurement setup described previously was employed to measure the response of GMR sensor to MNPs. The temperature of the GMR chips was controlled by means of a Peltier element coupled to the chip. The Peltier element was driven by a LFI3751 control unit (Wavelength Electronics, USA) with a Pt1000 thermoresistor. First, the chip was washed with 3 mL of 0.1% bovine serum albumin (BSA, Sigma Aldrich) and 0.05% Tween20 (Sigma Aldrich). The chip was then blocked with 100 μL of 1% BSA for 1 h in a shaker. After blocking, the chip was washed with 3 mL of 0.1% BSA and 0.05% Tween20, followed by washing with 3 mL of distilled water. Prior to sample injection, a base line signal measurement was performed for 2 min at 37° C. After sample injection, the DNA hybridization signals from different sensors were measured for 1 h at 37° C. Then, the temperature was lowered to 20° C. to inhibit further binding of the sample and stabilize DNA hybrids. The chip was washed five times with 100 μL of 0.05×SSC to remove unbound sample. 150 μL of 0.05×SSC were left in the reaction well and covered with 50 μL of mineral oil to prevent evaporation. Melting curves for all the probes were measured while temperature was ramped from 20° C. to 65° C. at 0.05° C./s. The temperature was then swept back to 20° C. to obtain temperature reference signals.

### Data Processing.

The MNPs are detected as a variation of the magnetoresistivity (ΔMR) of the GMR sensors. The temperature coefficients of ΔMR were calculated for each sensor as in Hall et al. using the temperature reference signals. A 5^{th} order polynomial was used to account for non-linear temperature dependency at high temperature. After temperature correction, the melting curves measured between 20° C. to 65° C. were normalized by their initial value at 20° C. The melting temperature Tₘ was defined as the temperature at which the normalized signal is 0.5. To calculate Tₘ, a first order polynomial was fitted to the melting curve in the region of interest. Each mutation (methylation) site was genotyped using two probes. ΔTₘ was defined as the difference between Tₘ measured for the probe complementary to the mutated (methylated) sequence minus Tₘ measured for the probe complementary to the Wild Type (un-methylated) sequence.

### Pyrosequencing.

The methylation status of the RARB and KIT promoter regions was analyzed by pyrosequencing using the PyroMark Q24 platform (Qiagen) and subsequent data analysis using the PyroMark Q24 software. Primer sequences are listed in Supplementary Table 3. DNA enzymatically methylated in vitro (CpGenome Universal Methylated DNA; Millipore) and unmethylated DNA prepared by whole genome amplification (WGA; GenomePlex, Sigma-Aldrich) was used as methylation-positive and -negative controls, respectively.

TABLE 1
List of ssDNA probes used for
mutation and methylation profiling.

| | | Tₘ | |
|---|---|---|---|
| GENE | Site | [° C.]* | Sequence** |
| BRAF Exon 11 | c.1391G > A | 44.7 | NH2-C6-5′- |
| NM_004333.4 | WT | | (9xT)AAATGATCCAGATCCAATTCTTTGTCC-3′ |
| | | 44.7 | NH2-C6-5′-(9xT) |
| | | | AATGATCCAGATTCAATTCTTTGTCCC-3′ |
| BRAF Exon 15 | c.1799T > A | 46.3 | NH2-C6-5′-(9xT) |
| NM_004333.4 | c.1798GT > AA | | CTCCATCGAGATTTCTCTGTAGCTAGAC-3′ |
| | WT | 46.6 | NH2-C6-5′-(9xT) |
| | | | TCCATCGAGATTTCTTTGTAGCTAGACC-3′ |
| | | 46.4 | NH2-C6-5′-(9xT) |
| | | | TCCATCGAGATTTCACTGTAGCTAGAC-3′ |
| NRAS Exon 2 | c.181C > A | 45.3 | NH2-C6-5′-(9xT) ACTGTACTCTTCTTTTCCAGCTGT-3′ |
| NM_002524.4 | c.182A > T | 45.5 | NH2-C6-5′-(9xT) ACTGTACTCTTCTAGTCCAGCTGTA-3′ |
| | WT | 45.5 | NH2-C6-5′-(9xT) CTGTACTCTTCTTGTCCAGCTGT-3′ |
| KIT Promoter | P1 Meth | 46.5 | NH2-C6-5′-(9xT) CCCAAAACCGCGAACGAC-3′ |
| | P1 uMeth | 46.4 | NH2-C6-5′-(9xT) CCCCCAAAACCACAAACAACAA-3′ |
| KIT Promoter | P2 Meth | 46.4 | NH2-C6-5′-(9xT) GAACGCGACAAAACCGAACC-3′ |
| | P2 uMeth | 46.5 | NH2-C6-5′-(9xT) ACAAACACAACAAAACCAAACCCC-3′ |
| RARB | P1 Meth | 44.0 | NH2-C6-5′-(9xT) |
| Promoter | P1 uMeth | 43.8 | ATCCTCAAACAACTCGCATAAAAAAATTC-3′ |
| | | | NH2-C6-5′- |
| | | | (9xT)AATCCTCAAACAACTCACATAAAAAAATTCT-3′ |
| RARB | P2 Meth | 45.6 | NH2-C6-5′-(9xT) GAATCCTACCCCGACGATACC -3′ |
| Promoter | P2 uMeth | 45.7 | NH2-C6-5′-(9xT) AAATCCTACCCCAACAATACCCA -3′ |
| Reference | Positive | | NH2-C6-5′-(9xT) TGC GAG CTT CGT ATT ATG GCG -3′ |
| | Negative | | TEG Biotin |
| | | | NH2-C6-5′-(9xT) GTGGGGCTAGGTG-3′ |

*Theoretical melting temperatures (Tₘ) were calculated with nearest neighbour (NN) model for 10 mM Na⁺ ionic concentration. Probes were designed to have matched Tₘ.
**All probes are amino-labelled to bind to GMR sensor surfaces.

TABLE 2
PCR primers for amplification
of EST cell line genomic DNA.

| | | Product |
|---|---|---|
| GENE | Sequence | length |
| BRAF Exon 11 | fw: biotin-C6-5′-TTGAC | 167 bp |
| NM_004333.4 | TTTTTTACTGTTTTTATC-3′ | |
| | bw: 5′-ATGTCACCACATTAC | |
| | ATACTTAC-3′ | |
| BRAF Exon 15 | fw: biotin-C6-5′-TTTTC | 167 bp |
| NM_004333.4 | CTTTACTTACTACACCTC-3′ | |
| | bw: 5′-GGAAAAATAGCCTCA | |
| | ATTCT-3′ | |
| NRAS Exon 2 | fw: biotin-C6-5′-CAAGT | 110 bp |
| NM_002524.4 | GGTTATAGATGGTGA-3′ | |
| | bw: 5′-AGGAAGCCTTCGCCT | |
| | GTCCT-3′ | |
| KIT | fw: biotin-C6-5′-GGGAG | 82 bp |
| Promoter* | GAGGGGTTGTTGTT-3′ | |
| | bw: 5′-TTCCAACTCTCCCCC | |
| | AAATACAAC-3′ | |
| RARB | fw: biotin-C6-5′-GGTTT | 179 bp |
| Promoter* | ATTTTTTGTTAAAGGGG-3′ | |
| | bw: 5′-AAAAATCCCAAATTC | |
| | TCCTTC-3′ | |

*KIT and RARB primers were designed to amplify bisulphite converted promoter region.

TABLE 3
Primers for pyrosequencing KIT
and RARB promoter regions of
bisulphite converted DNA from EST cell lines.

| Gene | Sequence |
|---|---|
| KIT | fw: 5′-GTGGAAAGGTGGAGAGAGAAA-3′ |
| Promoter | bw: biotin-5′-TTCCAACTCTCCCCCAAATACAAC-3′ |
| | S1: 5′-GAGGAGGGGTTGTTG-3′ |
| RARB | fw: biotin-C6-5′-GGTTTATTTTTTGTTAAAG |
| promoter | GGG-3′ |
| | bw: 5′-AAAAATCCCAAATTCTCCTTC-3′ |
| | S1: 5′-ACATCCCAATCCTCA-3′ |
| | S2: 5′-ATACTTACAAAAAACCTTCC-3′ |

TABLE 4
Parameters from linear fitting of ΔTₘ vs.
methylation density by pyrosequencing (FIG. 6A-C).
Numbers in parenthesis are standard errors on the last
digits from the fitting routine.

| | Slope | Intercept | |
|---|---|---|---|
| Location | [° C./%] | [° C.] | R² |
| KIT p1 | 0.22 (1) | −9 (1) | 0.97 |
| KIT p2 | 0.25 (1) | −8.8 (7) | 0.98 |
| RARB p1 | 0.075 (5) | −5.1 (2) | 0.97 |
| RARB p2 | 0.22 (2) | −9.3 (7) | 0.94 |

Above, we described the use of so-called planar Hall effect bridge (PHEB) sensors for real-time measurements of the temperature melting of DNA hybrids. We now describe examples of embodiments related to two-dimensional salt and temperature DNA denaturation analysis on a magnetoresistive sensor. In particular, these examples illustrate the combined effect of temperature and salt concentration and demonstrate two-dimensional salt and temperature denaturing mapping of a target to WT and MT probes to investigate salt concentration melting and to identify optimum conditions for discrimination between matching and mismatching target-probe hybrids. We further investigate the use of a single sensor bridge to discriminate between WT, MT and a mixture of these targets.

### Sensor Fabrication

The magnetoresistive sensor bridges were fabricated as described previously. Briefly, anisotropic magnetoresistive elements of nominal composition Ta(5 nm)/Ni₈₀Fe₂₀(30 nm)/Mn₈₀Ir₂₀(10 nm)/Ta(5 nm) were sputter-deposited in a saturating magnetic field. Electrical contacts of Ti(10 nm)/Pt(100 nm)/Au(100 nm)/Ti(10 nm) were deposited by electron beam evaporation. The sensors were spin coated with a 900 nm thick passivation layer (Ormocomp, Micro Resist Technology, GmbH, Germany).

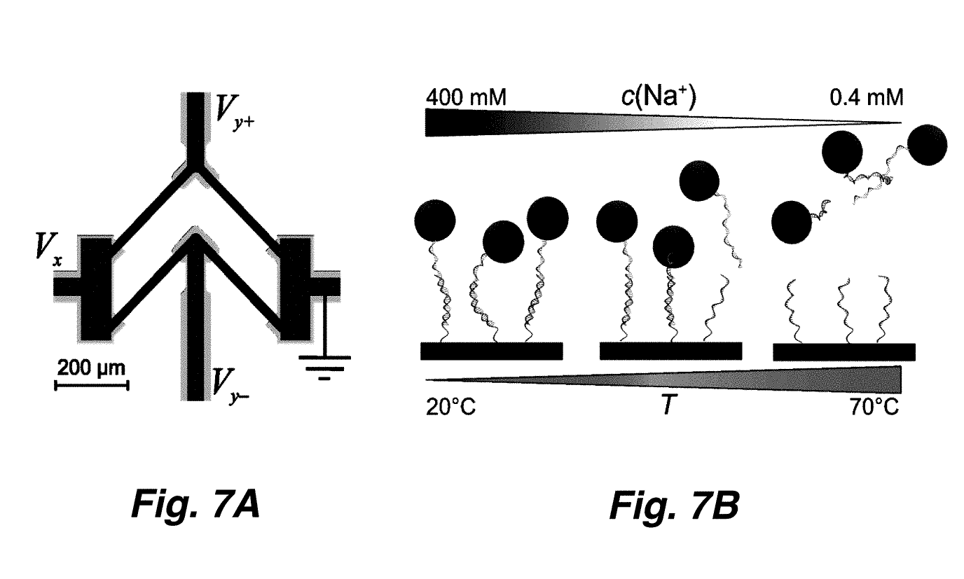

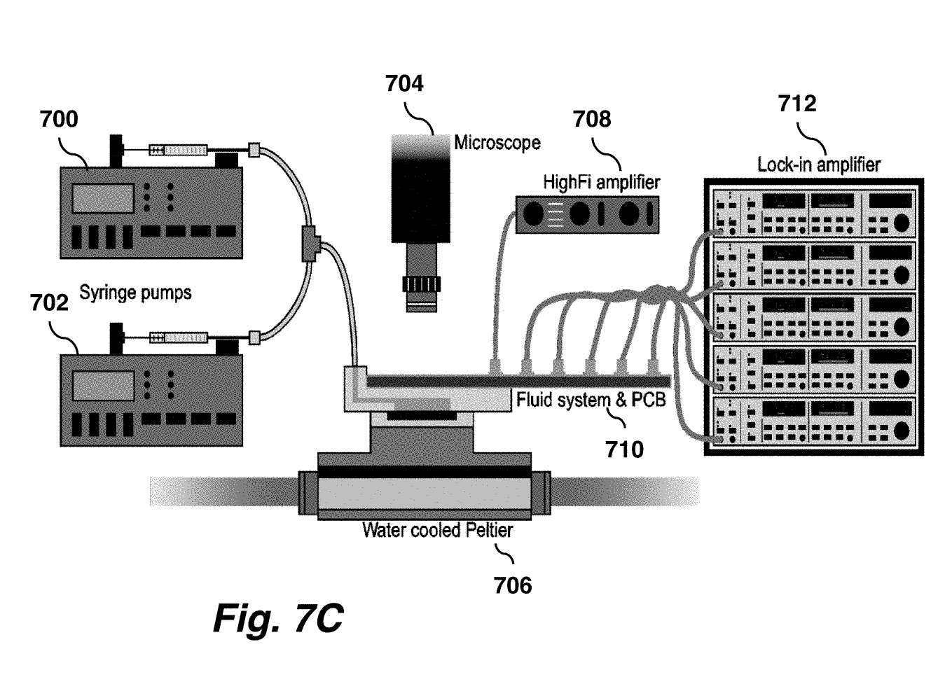

FIG. 7A is a schematic diagram of the differential magnetoresistive sensor bridge according to an embodiment of the invention. The magnetic material are diagonal bars connecting electrical contacts. Vₓ is the sensor bias voltage and V_{y} is the sensor bridge output voltage. The chip has five differential magnetoresistive sensor bridges, each having four sensor elements with length l=250 μm and width w=25 μm. This sensor design (termed differential planar Hall effect bridge, dPHEB) measures the differential signal between the top two branches and the bottom two branches of the bridge as described by Rizzi et al.

FIG. 7B is a schematic representation of temperature and salt concentration melting. Increasing temperature (bottom) or decreasing salt concentration (top) increases the stringency causing denaturation of the target-probe hybrids. Following denaturation, MNP-labeled targets are released from the sensor surface, resulting in a reduced signal from the sensor bridge.

### Measurement Platform

The measurement platform was previously described by Østerberg et al.¹⁷ and Rizzi et al.¹⁵ and is depicted in the schematic measurement setup of FIG. 7C. The chip is mounted in the microfluidic chamber below the circuit board that provides electrical connection to the chip via spring-loaded pins. The chip was mounted in a click-on microfluidic system 710 defining a fluidic channel (width×height×length=1 mm×1 mm×5 mm) over the sensor surface and providing electrical contact to the magnetoresistive sensors using spring-loaded pins.

A voltage of V_{RMS}=1.6 V at frequency f=167 Hz was applied to all sensor bridges connected in parallel using a commercial audio amplifier 708. The output voltage of each sensor bridge was measured using an SR830 Lock-In amplifier 712 with an SR552 preamplifier (Stanford Research Systems, Inc., USA). The MNPs were magnetized by the magnetic field due to the applied bias current through the sensor and the presence of MNPs on the sensor was detected in the imaginary part of the second harmonic lock-in signal. Microscope 704 is provided for imaging the surface of the chip.

The sensor chip was mounted in an aluminum chip holder with good thermal contact. The temperature of the chip mount was measured with a Pt1000 thermometer, and controlled via a LFI3751 control unit (Wavelength Electronics, USA) driving a Peltier element. The other side of the Peltier element was cooled using a commercial CPU water cooling system 706.

Two syringe pumps 700, 702 (model 540060, TSE systems, Germany), connected to a chip inlet via a T-branch, provided the liquid flow in the chip during washing. They were controlled via a custom LabView program such that any ratio of the two liquids could be injected while maintaining a constant total liquid flow rate.

### Sensor Functionalization

The sensor elements on each of the five sensor bridges could be selectively functionalized with amino modified ssDNA probes as described by Rizzi et al. The probes to genotype SNPs in the human beta globin (HBB) gene (sequences in Supplementary Information) were adapted from Petersen et al. and were purchased from DNA Technology A/S, Denmark. One of the sensor bridges on each chip was used as a positive reference and was functionalized on its top half with a biotinylated DNA probe. Two sensor bridges were used for direct detection of the wild type (WT) or mutant type (MT) variants of the CD 8/9 locus of the HBB gene and were functionalized on their top halves with the corresponding respective probes. We will refer to these as the MT and WT sensors, respectively. To perform WT-MT differential detection of the CD 8/9 and CD 17 loci of the HBB gene, two sensor bridges on a chip were functionalized on their top and bottom halves with probes matching the WT and MT variants, respectively.

### Hybridization.

The solution of target-labeled MNPs was prepared by mixing a solution of 10 nM biotinylated target DNA (sequences in supplementary information) in 4× Saline Sodium Citrate (SSC, Gibco, USA) buffer 1:1 v:v with the stock solution of Miltenyi Streptavidin Microbeads (Miltenyi Biotec Norden AM, Sweden) to a final target concentration of 5 nM (buffer salt concentration c(Na⁺)=400 mM). The target-MNP solution was injected over the sensors and incubated for 30 min at T=37° C.

### Temperature Melting.

After hybridization of WT DNA target, the chip was washed with diluted SSC buffer to a final concentration c(Na⁺)=10 mM or 2 mM at T=20° C. for 80 s at 30 μL/min. Following washing, the temperature was ramped from T=20° C. to 70° C. at 0.1° C./s. The melting data was corrected for the temperature dependence of the sensor sensitivity using a reference sweep from T=70° C. to 20° C. measured after complete denaturation of the hybrids as described previously.

### Salt Concentration Melting.

After hybridization of WT DNA target, the chip was washed with 2×SSC (c(Na⁺)=400 mM) at T=30° C. or 40° C. at 30 μL/min for 80 s. After this initial washing, the washing buffer concentration was varied exponentially from c(Na⁺)=400 mM to 0.4 mM over 1200 s by mixing the flow of 2×SSC buffer from syringe pump one with milliQ water from syringe pump two. Concentration dependent sensor offsets determined from a reference concentration profile measurement with no MNPs were subtracted from the data.

### WT-MT Differential Measurements.

Three 5 nM target DNA solutions were analyzed on the sensor bridges functionalized for WT-MT differential detection: WT, MT and 1:1 WT:MT. After hybridization, melting curves were measured during both temperature and salt concentration melting. Temperature melting was performed as described above with c(Na⁺)=10 mM. Salt concentration melting was performed as described above at T=37° C. The signal measured during temperature melting was corrected for the temperature dependence of the sensor sensitivity and normalized by the signal from the positive reference sensor functionalized with biotinylated DNA.

### Temperature Melting

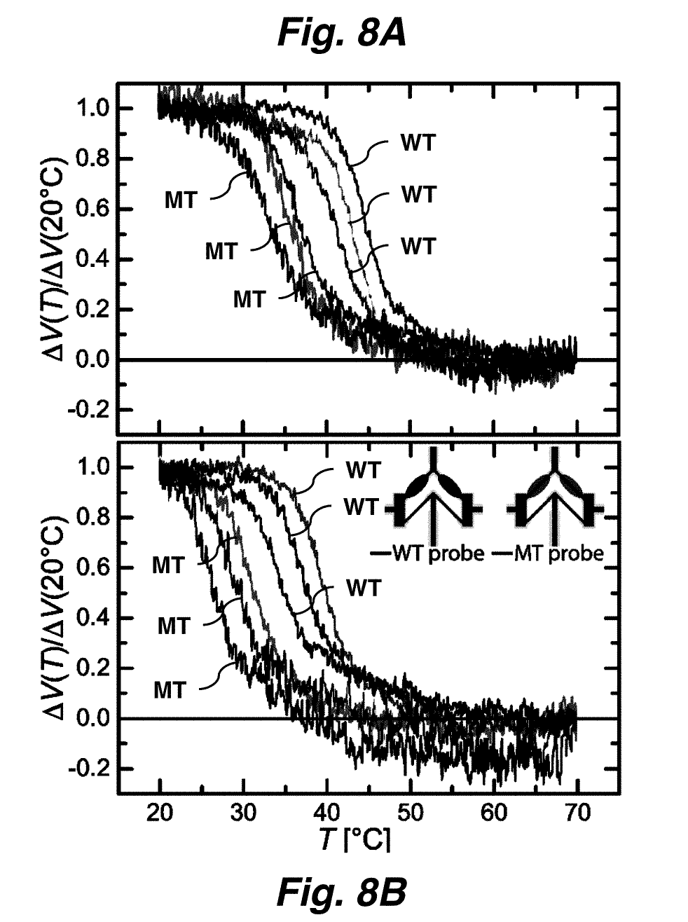

FIG. 8A, FIG. 8B show the WT target melting curves measured for c(Na⁺)=10 mM and 2 mM, respectively of MNP labeled WT DNA target hybridized to WT and MT DNA probes for the CD8/9 mutation. The real-time data was corrected for the temperature dependence of the sensor output and normalized to the initial value at 20° C. The two sensor bridges were functionalized as depicted in the inset. The DNA hybrids were denatured by increasing the temperature from T=20° C. to 70° C. at 0.1° C./s for FIG. 8A c(Na⁺)=10 mM and FIG. 8B c(Na⁺)=2 mM. Results of triplicate experiments are shown.

Biotinylated WT DNA target, labeled with streptavidin MNPs, at a DNA concentration of c=5 nM in 2×SSC buffer (c(Na⁺)=400 mM) was incubated over the sensors at T=37° C. for 30 min. The measurements below were performed with the WT and MT sensors for SNP detection of the CD 8/9 locus in HBB (see inset in FIG. 8B). The signal due to MNPs was measured in the imaginary part of the 2^{nd} harmonic bridge voltage, here written as V, in response to the AC bias of the bridge. When the top and bottom halves of the bridge experience the same concentration and distribution of MNPs, the bridge is balanced and nominally zero signal is detected. Hybridization of MNP-labeled DNA to the top half of a sensor bridge increases the local MNP concentration at this half of the sensor bridge and causes an increase of V.

After hybridization, the chip temperature was decreased to T=20° C. to stabilize hybrids and inhibit further binding. Unbound target-MNPs were removed by washing. Two buffers were tested, with c(Na⁺)=10 mM and 2 mM, respectively. Following washing, the buffer was left stagnant over the sensors and the temperature was ramped from T=20° C. to 70° C. at 0.1° C./s to measure the melting of the DNA hybrids in real time.

For c(Na⁺)=10 mM (FIG. 8A) the signals for both the WT and MT sensors were stable between 20° C. and 30° C. At T>30° C., the signal from the MT sensor decreased sharply indicating a temperature melting of the DNA hybrids. For the WT sensor, the melting was shifted to a higher temperature. Some variation in the absolute melting temperatures was observed between the three experiments. However, in each experiment, we found the temperature shift between the MT and WT sensors to be reproducible and with a value of about 8° C.

For c(Na⁺)=2 mM (FIG. 8B) the results followed a similar trend. The MT sensor signal showed a sharp decrease at a temperature, which was about 9° C. lower than for the WT signal. Compared to FIG. 8A, the melting also took place at lower temperature.

### Salt Concentration Melting

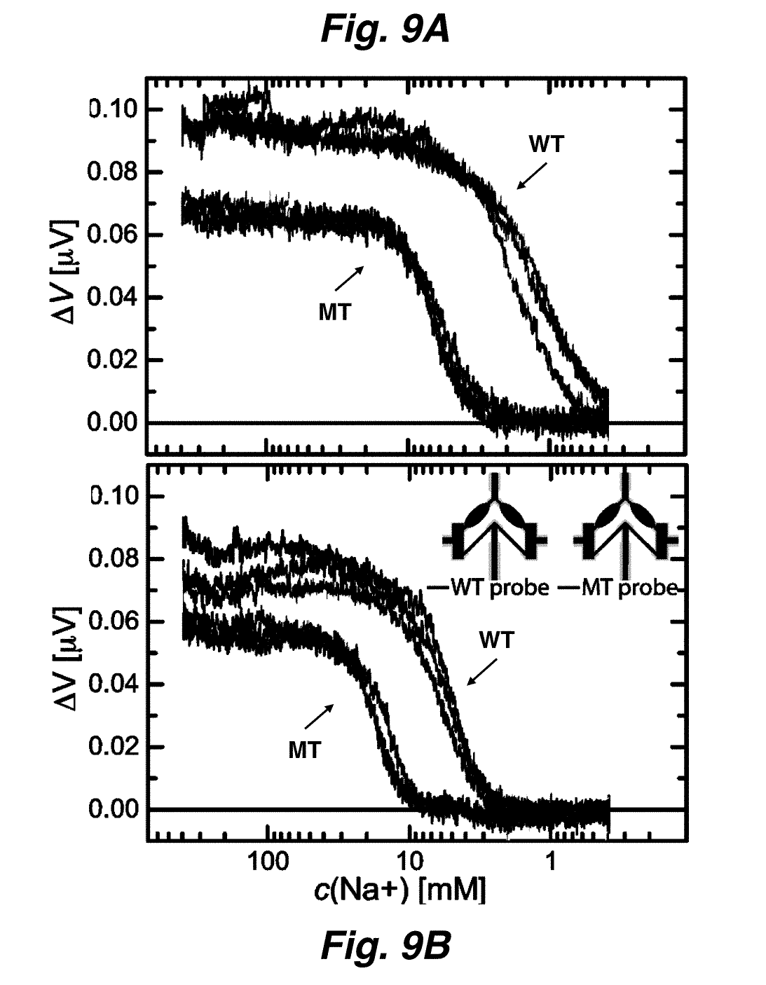

FIG. 9A, FIG. 9B show salt concentration melting curves of WT DNA target hybridized to WT and MT DNA probes for the CD8/9 mutation. PHEB sensors were functionalized as depicted in the inset. The hybrids were denatured by decreasing the salt concentration from c(Na⁺)=400 mM to 0.4 mM over 1200 s at T=30° C. (FIG. 9A) and T=40° C. (FIG. 9B). Results of triplicate experiments are shown.

Melting measurements of the MNP-labeled WT DNA hybrids were performed at fixed temperature as function of decreasing salt concentration in the washing buffer. Following hybridization, the sensor temperature was set to 30° C. or 40° C. and unbound labels were washed off with 2×SSC buffer (c(Na⁺)=400 mM) at a flow rate of 30 μL/min. Subsequently, the washing buffer salt concentration was exponentially decreased from c(Na⁺)=400 mM to 0.4 mM over 1200 s while maintaining a constant total flow rate of 30 μL/min by varying the relative flow rate of the two syringe pumps loaded with 2×SSC and MilliQ water, respectively. Salt concentration melting curves were measured in real-time during the decreasing concentration profile. The results are shown in FIG. 9A, FIG. 9B obtained at T=30° C. and 40° C.; a logarithmic time scale is used because of the exponential time profile of the buffer concentration. The final value of the sensor signal at c(Na⁺)=0.4 mM was subtracted to obtain the signal variation ΔV(c).

At T=30° C. (FIG. 9A), the signals for both WT and MT sensors were approximately constant at high salt concentrations, c(Na⁺)>20 mM, and the WT sensor signal was 25% higher than that from the MT sensor. The three experiments were highly reproducible and therefore no normalization of the data was performed. The MT sensor signal decreased sharply between c(Na⁺)=20 mM and 2 mM indicating a melting of the DNA hybrids. The signal from WT probe decreased at a lower salt concentration c(Na⁺)<3 mM.

At T=40° C. (FIG. 9B), the same trend was observed, but the melting curves were shifted to higher salt concentrations, such that melting took place at higher c(Na⁺) compared to T=30° C.

Melting Temperature Tₘ and Concentration cₘ

Error function fits to the temperature melting curves in FIG. 8A, FIG. 8B were performed to extract the melting temperature Tₘ defined as the temperature at which the curve reached 50% of its initial value. For c(Na⁺)=10 mM we obtained Tₘ(WT)=43±1° C. and Tₘ(MT)=35±1° C. (uncertainties indicate standard error of the mean (SDOM), n=3) for the WT and MT sensors, respectively. The corresponding values for c(Na⁺)=2 mM were Tₘ(WT)=38±1° C. Tₘ(MT)=29±1° C.

Similarly, error function fits to the data in FIG. 9A, FIG. 9B vs. log(c) were performed to extract the melting concentration cₘ, defined as the point at which the error function reached 50% of the initial value. At T=30° C. we obtained cₘ(WT)=1.4±0.1 mM and cₘ(MT)=6.3±0.3 mM and at T=40° C. we obtained cₘ(WT)=5.7±0.2 mM and cₘ(MT)=15±1 mM. Uncertainties indicate SDOM (n=3).

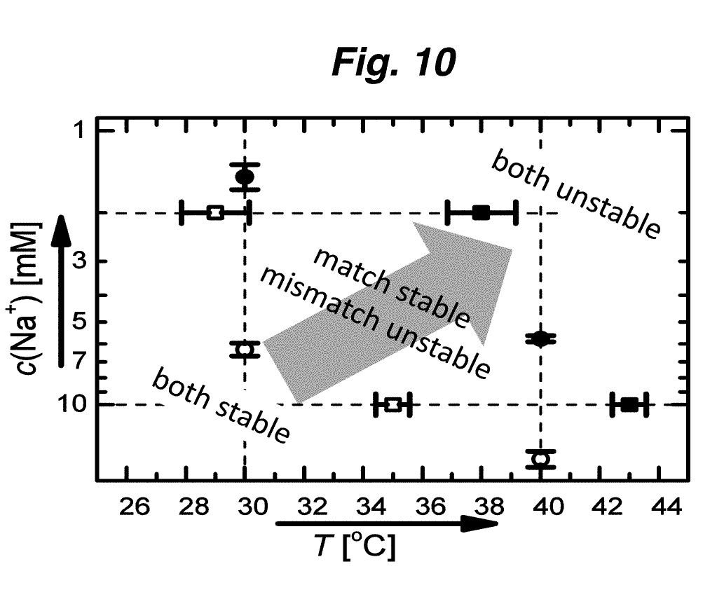

FIG. 10 shows the values of melting temperature Tₘ and salt concentration cₘ obtained from error function fits to the temperature and salt melting, respectively, for the WT target and WT probes (filled symbols) and MT probes (open symbols) for the CD 8/9 locus of HBB. Dashed lines represent the temperature (horizontal) and concentration (vertical) profiles used. The arrow indicates direction of increasing stringency. Error bars are standard error of the mean (n=3). The values of Tₘ and cₘ obtained from the denaturation experiments are collected in FIG. 10. The dashed lines represent the temperature or concentration profiles used. The perfectly matched WT probe-WT target gave denaturation points at higher stringency compared to the mismatched MT probe-WT target hybrids. The separation between the two is not constant, but it is maximal in the central region of the plot.

### Genotyping Using WT-MT Differential Measurements

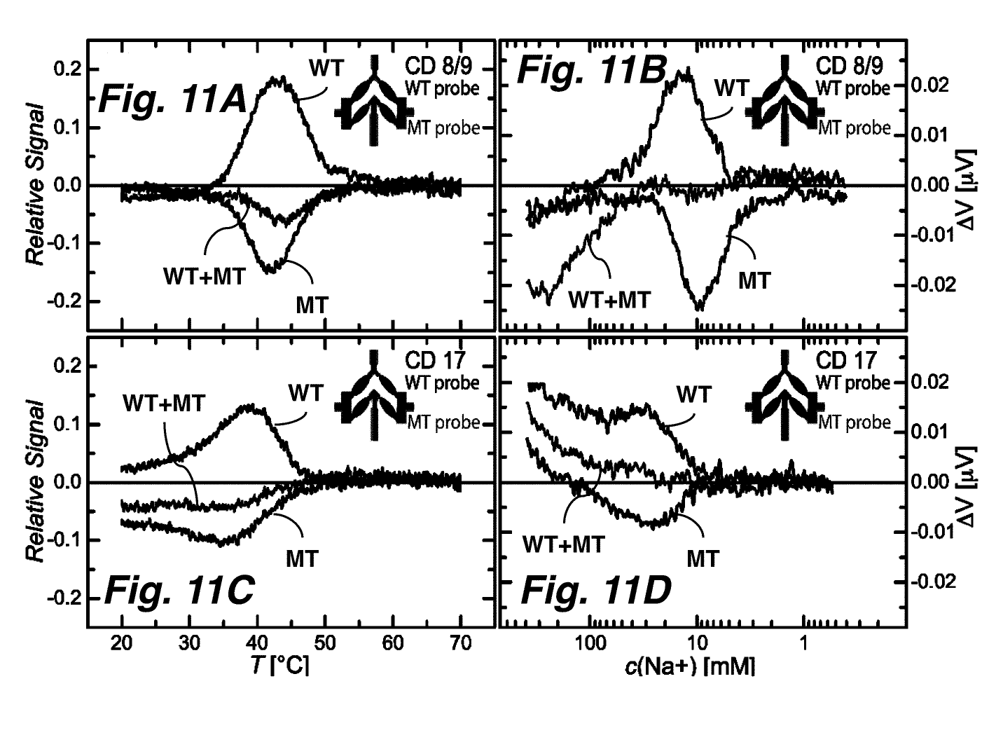

We have previously shown that a single sensor bridge can be used for genotyping when its top and bottom halves are functionalized with WT and MT probes, respectively. The sensor output is proportional to the difference in the amount of MNPs bound to the top and bottom halves of the sensor bridge. To genotype each of the CD 8/9 and CD 17 loci of the HBB gene with respect to the mutations given in Table 1, we functionalized a sensor bridge with WT and MT probes on its top and bottom halves, respectively (see insets in FIG. 11A-D). Three target combinations were measured: WT, MT and 1:1 WT:MT.

FIG. 11A shows the temperature melting curve measured at c(Na⁺)=10 mM for the CD 8/9 locus. Note that the curves show the signal relative to that obtained from the positive reference sensor. At low temperature, the relative signal was close to zero for all three targets, indicating identical hybridization of the targets to both WT and MT probes. At temperatures T=30° C. to 50° C., the relative signal from the three target clearly differed from each other. For the WT target, the relative signal peaked at a positive value of 0.17 whereas for the MT target, the signal peaked at a negative value of −0.15. The signal from the 1:1 WT:MT target mixture maintained a value closer to zero and reached a minimum value of −0.06. Above T=50° C. the relative signal for the three targets stabilized at zero.

FIG. 11B shows the salt concentration melting curve measured at T=37° C. for the CD 8/9 locus. At high salt concentration (c(Na⁺)>50 nM) the WT and MT targets showed similar values in the range ΔV=−0.005 μN to 0 μV. The signal from the mixed WT:MT target showed a lower value (ΔV=−0.021 μV) that increased towards zero for increasing c(Na⁺). For c(Na⁺)=40 nM to c(Na⁺)=4 nM, the three targets showed maximum separation, with the WT target reaching ΔV=+0.023 μV, the MT target reaching ΔV=−0.023 μV, and the mixed MT:WT target showing a stable signal near ΔV==0 μV. At low salt concentration (c(Na⁺)<4 nM) the signals from the three targets stabilized at zero.

FIG. 11C, FIG. 11D show the corresponding temperature and salt concentration melting profiles measured for the CD 17 locus. In the temperature melting study (FIG. 11C), the three targets showed a clear separation even at low temperature. The trend with temperature was the same as for the CD 8/9 locus except that the peaks appeared at lower temperatures and the peak levels were slightly lower. The maximum separation between the three targets was observed between 35° C. and 40° C. In the salt concentration melting study (FIG. 11D), the three targets were initially separated at high c(Na⁺) with ΔV(WT)>ΔV(WT:MT)>ΔV(MT)>0. Upon increasing stringency (decreasing c(Na⁺)), ΔV for all three targets decreased and separated more from each other. The largest separation was observed for c(Na⁺)=29 mM and corresponded to ΔV(MT)=+0.015 μV, ΔV(MT)=−0.008 μV, and ΔV(WT:MT)=+0.002 μV. For c(Na⁺)<7 mM, the signal from all targets approached ΔV=0.

### Melting Curves Discussion

The differential design of the bridge sensors allowed for real-time measurement of the formation and melting of DNA hybrids. The integration of the sensor chip in a microfluidic system with temperature control allowed us to perform two-dimensional melting studies as function of temperature and/or salt concentration. Both of these two parameters affect the stability of DNA hybrids and can thus be used to discriminate between perfectly matched target-probe hybrids and mismatches down to a single nucleotide level. The magnetic sensor platform is compact and sufficiently robust to enable readout under conditions of varying temperature or salt concentration. Both temperature and salt concentration melting curves were measured to characterize the investigated loci of the HBB gene. In both investigations, the unmatched MT probe-WT target duplexes denatured at lower stringency (lower T, higher c(Na⁺)) compared to the perfectly matched counterparts. As expected from nearest neighbor models, decreasing buffer c(Na⁺) leads to lower Tₘ for both matched and mismatched hybrids. Similarly, increasing T leads to higher cₘ.

FIG. 11A-D Melting curves (FIG. 11A, FIG. 11C) and salt concentration denaturation curves (FIG. 11B, FIG. 11D) measured on differential sensors for WT, MT, and a 1:1 WT:MT mixture of target DNA. The sensors were functionalized with WT and MT probes for the CD 8/9 locus (FIG. 11A,B) or the CD 17 locus (FIG. 11C, FIG. 11D) as indicated in the insets.

The two methods showed clear differences in reproducibility. While salt concentration denaturation curves (FIG. 9A, FIG. 9B) were almost perfectly overlapping, the absolute melting temperatures measured for repeated measurements showed an uncertainty of about 2° C. (SDOM, n=3). The differences in the simultaneously measured melting temperatures measured for the two sensors, ΔTₘ=Tₘ(WT)−Tₘ(MT), were found to ΔTₘ=7.7±0.2° C. (c(Na⁺)=10 mM) and 8.9±0.5° C. (c(Na⁺)=2 mM), where the stated uncertainties are SDOM (n=3). Thus, the uncertainty on ΔTₘ was significantly lower than that on the absolute melting temperature. This indicates that it was difficult to accurately replicate identical temperature profiles. The variation in the absolute temperature may, for example, originate from differences in temperature of the washing buffer or the chip surroundings. Our results further indicate that salt concentration profiles are more easily reproduced. Moreover, the signal from the magnetoresistive sensors is only weakly sensitive to a change in the salt concentration. Further, the requirements for temperature control are less restrictive for salt concentration melting and the concentration can be varied using a simple two-pump setup. Therefore, concentration melting experiments are easier to implement.

Two-Dimensional Mapping of Tₘ and cₘ

The measurements of the melting curves as function of both temperature and salt concentration allowed us to map the point of denaturation over these two dimensions of experimental conditions. The resulting map of FIG. 10 presents the measured Tₘ and cₘ. These results offer a non-trivial insight into the stability of DNA hybrids over a wide range of stringencies in the (T, c(Na⁺))-plane.

Hybridization-based assays aim to discriminate between matched and mismatched probe-target hybrids. In an end-point detection scheme, this is done by selecting a stringency condition that, to the extent possible, denatures mismatched hybrids and maintains the matched hybrids. Similarly, a denaturation assay should maximize the gap between the melting points of the matched and mismatched hybrids. In FIG. 10 we can identify an optimal region between T=32-38° C. and c(Na⁺)=3-7 mM where the distance between open and solid symbols is maximal.

As a perspective, the presented magnetoresistive sensor detection scheme and setup allows for any profile in the (T, c(Na⁺))-plane, i.e., for a simultaneous change of T and c(Na⁺). FIG. 10 indicates that a potentially better separation between WT and MT could be obtained along the diagonal in the (T, c(Na⁺))-plane by simultaneously ramping the temperature up and the salt concentration down. This will be topic for future investigation.

Magnetoresistive sensor arrays with up to thousand sensors have been presented in the literature.¹⁶ Real-time two-dimensional maps of the melting of a DNA target hybridized to an array of WT and MT detection probes enable a highly parallel screening of conditions for a range of sequences and probe lengths to target a number of loci and genes in a DNA target. This can be used for direct genotyping but also to identify regions on the (T, c(Na⁺))-plane that are optimal for end-point detection after a stringent washing. This could significantly ease the design and improve performance of microarrays, where the probe design may be challenging as a single stringent wash has to produce a large difference between matching and mismatching hybrids while still maintaining a significant signal from the matching hybrids.

### Genotyping Using WT-MT Differential Measurements

By functionalizing the top and bottom halves of a single sensor bridge with WT and MT probes, it was possible to measure the differential binding of target to the two probes. For a given sensor array, this configuration allows for the investigation of a higher number of mutation sites, since only a single sensor is used for each mutation.

The melting curves of FIG. 11A, FIG. 11B showed different behaviors for the CD 8/9 and CD 17 loci. The stability of the target-probes hybrids depends on the length of the probe, its C+G content and the type of mutation investigated. Here, the mutation at the CD 8/9 locus is a single base (C) insertion, whereas that at the CD 17 locus is a single base (T>A) transversion. The probes had lengths of 22 bases (C+G 54%) and 18 bases (C+G 66%) for the CD 8/9 and CD 17 loci, respectively.

The shorter probe length for CD 17 caused a separation of the three targets also at low stringency (FIG. 11C, FIG. 11D) and maximum separation between the targets was found at lower stringencies compared to CD 8/9. Moreover, the base insertion in CD 8/9 resulted in more unstable mismatched hybrids and caused a higher separation between the three targets (FIG. 11A, FIG. 11B).

For the CD 8/9 locus, the initial negative signal from the 1:1 WT:MT mixed target at high c(Na⁺) in FIG. 5b is likely caused by a higher affinity of the MT target-MT probe hybrids compared to that of the WT target-MT probe. This is supported by the observation that the signal from the MT target peaks at lower c(Na⁺) (higher stringency) than the WT target in FIG. 11B.

For the salt concentration melting for the CD 17 locus (FIG. 11D), the initial signals from all three targets at high c(Na⁺) were positive and were found to decrease with decreasing salt concentration. We speculate that this is caused by higher unspecific binding to the WT probe than the MT probe.

The different behavior of the WT and MT probes for the CD 8/9 and CD 17 loci would require optimization of the assay in end-point detection to determine the optimum washing stringency to perform a correct genotyping. Instead, using a denaturation curve method, the hybrids are subject to a continuously varying stringency and thus we could easily differentiate the three different target compositions.

### CONCLUSION

The examples above demonstrated the use of a magnetoresistive sensor array integrated in a lab-on-a-chip system for studies of the denaturation of DNA hybrids as function of both temperature and salt concentration. The magnetic readout was only weakly sensitive to the varying experimental conditions and could therefore be used to provide a sensitive real-time readout of the signal from the magnetic nanoparticle labeled DNA target hybridized to detection probes. The differential sensor design enabled studies of the specific binding of a WT target to WT and MT detection probes for two loci of the human HBB gene. Melting experiments at different cuts in the temperature-salt concentration plane identified a region of optimal discrimination between the two. Further, it was found that salt concentration melting curves were more reproducible than temperature melting curves. These provide a hitherto not studied but interesting alternative to temperature melting curves in lab-on-a-chip systems.

Further, the examples demonstrated the discrimination between WT, MT and 1:1 WT:MT targets using a single sensor bridge functionalized on its top and bottom parts with WT and MT probes, respectively. This was performed both for temperature melting and salt concentration melting.

This demonstrates the feasibility of using a lab-on-a-chip magnetoresistive sensor arrays for the characterization of the stability of DNA hybrids as function of both salt concentration and temperature. On a larger sensor array, this can be used for simultaneous mapping of a number of probe-target interactions in the temperature-salt concentration plane for real-time detection or to identify regions of optimal assay conditions.

1. A method of methylation detection that provides a quantitative description of the methylation density in DNA strands, the method comprising:
performing bisulphite conversion of the DNA strands containing methylated and unmethylated sites to create converted DNA strands with mismatch base pairs;
performing PCR amplification of the converted DNA strands to produce PCR amplified converted DNA strands;
magnetically labeling the PCR amplified converted DNA strands;
hybridizing PCR amplified converted DNA strands to complementary DNA strands immobilized onto a magnetoresistive (MR) sensor array, wherein the hybridizing is performed before or after the magnetically labeling;
increasing a stringency condition to cause the magnetically labeled single strand target DNA strands to be denatured from the complementary DNA strands immobilized onto a magnetoresistive (MR) sensor array;
reading out in real time during the increasing of the stringency condition a denaturation signal resulting from the denatured magnetically labeled single strand target DNA strands;
determining stringency conditions of methylated and unmethylated DNA strands from the denaturation signal.

2. The method of claim 1 further comprising reading out in real time a binding signal during hybridizing the magnetically labeled single strand target DNA strands with complementary DNA strands immobilized onto a magnetoresistive (MR) sensor array.

3. The method of claim 2 wherein the stringency condition is temperature, wherein increasing the stringency condition comprises increasing the temperature while salt concentration is held constant, and wherein determining the stringency conditions of the methylated and unmethylated DNA strands comprises determining melting temperatures of the methylated and unmethylated DNA strands.

4. The method of claim 2 wherein the stringency condition is salt concentration, wherein increasing the stringency condition comprises decreasing the salt concentration while temperature is held constant, and wherein determining the stringency conditions of the methylated and unmethylated DNA strands comprises determining melting salt concentrations of the methylated and unmethylated DNA strands.

5. The method of claim 3 wherein performing bisulphite conversion of the DNA strands containing methylated and unmethylated sites comprises performing bisulphite conversion of the DNA strands containing methylated and unmethylated sites and wild type genes and mutated genes; and wherein determining stringency conditions of methylated and unmethylated DNA strands from the denaturation signal comprises determining stringency conditions of methylated and unmethylated DNA strands and wild type genes and mutated type genes from the denaturation signal, whereby mutation sites may be determined simultaneously with methylation sites.

6. A method of methylation detection that provides a quantitative description of the methylation density in DNA sequences, comprising: Bisulphite conversion of DNA strands with or without methylated sites; PCR amplification of converted DNA strands; Hybridization of converted target DNA strands with a MR sensor array immobilized with (unmethylated) complementary DNA strands; Adding methyltransferase to methylate the complementary DNA strands corresponding to the methylated sites of the target DNA strands; Ramping up temperature until target DNA strands are denatured from the immobilized DNA strands, leaving behind the methylated single strand DNA if the target DNA is methylated, or leaving behind the unmethylated single strand DNA if the target DNA is unmethylated; Adding magnetic nanoparticles conjugated with methyl-recognizing moieties, such as antimethylated lysine antibody, which will bind to methylated DNA strands immobilized on the sensor; Reading out the binding signal in real time, and determining if the immobilized DNA strand (and thus the corresponding target DNA strand) is methylated or not.

7. The method of claim 4 wherein performing bisulphite conversion of the DNA strands containing methylated and unmethylated sites comprises performing bisulphite conversion of the DNA strands containing methylated and unmethylated sites and wild type genes and mutated genes; and wherein determining stringency conditions of methylated and unmethylated DNA strands from the denaturation signal comprises determining stringency conditions of methylated and unmethylated DNA strands and wild type genes and mutated type genes from the denaturation signal, whereby mutation sites may be determined simultaneously with methylation sites.
